(19) United States
(12) Patent Application Publication
(10) Pub. No.: US 2024/0293253 A1
(43) Pub. Date: Sep. 5, 2024

(54) NONINVASIVE UNISEX EXTERNAL CATHETER

(71) Applicants: Victor Musoke, Van Nuys, CA (US); Sam Ristich, Los Angeles, CA (US)

(72) Inventors: Victor Musoke, Van Nuys, CA (US); Sam Ristich, Los Angeles, CA (US)

(21) Appl. No.: 18/117,389

(22) Filed: Mar. 3, 2023

(51) Int. Cl. A61F 5/451; A61F 5/44; A61F 5/441

(52) U.S. Cl. CPC A61F 5/451; A61F 5/4405; A61F 5/441

(57) ABSTRACT

A unisex external catheter comprising of a noninvasive flexible body that contours to different shapes and sizes. The noninvasive external catheter provides greater comfort, prevents urine leakage, can be used for both men and women and is easier to use since it does not require puncturing or piercing a natural human orifice.

### BACKGROUND

Current measures for bladder control are evasive, leave clients vulnerable to urinary tract and skin infections, require frequent change management, and are prone to leakages.

### FIELD OF INVENTION

The present invention is related to noninvasive urinary evacuation and more particularly concerns an external catheter for both men and women.

### SUMMARY OF THE INVENTION

A noninvasive unisex external catheter will now be described in accordance with one or more embodiments of the invention. The following exemplary description numerous specific details are set forth to provide a more thorough understanding of embodiments of the invention. Furthermore, although steps or processes are set forth in an exemplary order to provide an understanding of one or more systems and methods, the exemplary order is not meant to be limiting. One of ordinary skill in the art would recognize that the steps or processes may be performed in a different order, and that one or more steps or processes may be performed simultaneously or in multiple process flows without departing from the spirit or the scope of the invention. In other instances, specific features, quantities, or measurements well known to those of ordinary skill in the art have not been described in detail so as not to obscure the invention. It should be noted that although examples of the invention are set forth herein, the claims, and the full scope of any equivalents, are what define the metes and bounds of the invention.

For a better understanding of the disclosed embodiment, its operating advantages, and the specified object attained by its uses, reference should be made to the accompanying drawings and descriptive matter in which there are illustrated exemplary disclosed embodiments. The disclosed embodiments are not intended to be limited to the specific forms set forth herein. It is understood that various omissions and substitutions of equivalents are contemplated as circumstances may suggest or render expedient, but these are intended to cover the application or implementation.

The term “first”, “second” and the like, herein do not denote any order, quantity or importance, but rather are used to distinguish one element from another, and the terms “a” and “an” herein do not denote a limitation of quantity, but rather denote the presence of at least one of the referenced items.

Spatially relative terms, such as “beneath,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

It will be understood that when an element or layer is referred to as being “on,” “connected to,” or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively. Also, the term “exemplary” is intended to refer to an example or illustration. Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Disclosed in this application is an external catheter comprising a) a body, wherein said body is a hollow concave shape, b) a lip, wherein said lip is attached around perimeter of said body, c) a silicone layer, wherein said silicone layer is a single layer of said body, d) a drain element, wherein said drain element is configured for urine drainage, e) a an outlet, wherein said outlet covers said drain element, f) a fiber element, wherein said fiber element is attached to said body, wherein said fiber element abuts to drain element, g) a collection region, wherein said collection region abuts to said fiber element, h) a vent, wherein said vent abuts to said body, and i) a valve, wherein said valve is attached to said vent. In addition, the external catheter is unisex. The silicone layer c) wherein said silicone layer enables said body to be elastic, wherein said elasticity enables said body to contour to a plurality of human bodies. The collection region g) wherein said fiber element guides a stream of urine to said drain element, wherein said stream of urine is collected for disposal. In addition, the valve i) wherein said valve is configured to allow air inside said body, wherein said valve does not allow liquid outside said body.

In another embodiment is an external catheter comprising a) a body, wherein said body is a hollow concave shape, b) a lip, wherein said lip is attached around perimeter of said body, c) an adhesive element, wherein said adhesive element is attached to said lip, wherein said lip and said adhesive element form a border such that said border enables a sealed attachment to a human body, d) a silicone layer, wherein said silicone layer is a single layer of said body, e) a drain element, wherein said drain element is configured for urine drainage f) an outlet, wherein said outlet covers said drain element, g) a fiber element, wherein said fiber element is attached to said body, wherein said fiber element abuts to drain element, h) a collection region, wherein said collection region abuts to said fiber element, i) a vent, wherein said vent abuts to said body, and j) a valve, wherein said valve is attached to said vent. In addition, the external catheter is unisex. The silicone layer d) wherein said silicone layer enables said body to be elastic, wherein said elasticity enables said body to contour to a plurality of human bodies. The collection region h) wherein said fiber element guides a stream of urine to said drain element, wherein said stream of urine is collected for disposal. In addition, the adhesive element c) wherein said adhesive element prevents urine from leaking outside said body during urination. The valve j) wherein said valve is configured to allow air inside said body, wherein said valve does not allow liquid outside said body.

In another embodiment is an external catheter comprising a) a body, wherein said body is a hollow concave shape, b) a lip, wherein said lip is attached around a perimeter of said body, c) a silicone layer, wherein said silicone layer is a single layer of said body, d) a drain tube, wherein said drain tube is configured for urine drainage, e) an outlet, wherein said outlet covers said drain tube, f) a cotton material, wherein said cotton material is attached to said body, wherein said cotton material abuts to drain tube, g) a collection region, wherein said collection region abuts to said cotton material, h) a vent, wherein said vent abuts to said body, i) a valve, wherein said valve is attached to said vent and j) an attachment element, wherein said attachment element is configured to hold said body, wherein said attachment element is adjustable. In addition, the silicone layer c) wherein said silicone layer enables said body to be elastic, wherein said elasticity enables said body to contour to a plurality of human bodies. The collection region g), wherein said cotton material guides a stream of urine to said drain tube, wherein said stream of urine is collected for disposal. The cotton material f), wherein said cotton material is placed inside said outlet. The valve i), wherein said valve is configured to allow air inside said body, wherein said valve does not allow liquid outside said body.

In another embodiment is an external catheter comprising a) a body, wherein said body is a hollow concave shape, b) a lip, wherein said lip is attached around a perimeter of said body, c) a silicone layer, wherein said silicone layer is a single layer of said body, d) a drain tube, wherein said drain tube is configured for urine drainage, e) an outlet, wherein said outlet covers said drain tube, f) a cotton material, wherein said cotton material is attached to said body, wherein said cotton material abuts to drain tube, g) a collection region, wherein said collection region abuts to said cotton material, h) a vent, wherein said vent abuts to said collection region, i) a valve, wherein said valve is attached to said drain element, j) an attachment element, wherein said attachment element is configured to hold said body, wherein said attachment element is adjustable, and k) an underwear, wherein said underwear is configured with said attachment element to hold said body, wherein said underwear is unisex. In addition, the silicone layer c), wherein said silicone layer enables said body to be elastic, wherein said elasticity enables said body to contour to a plurality of human bodies. The collection region g), wherein said cotton material guides a stream of urine to said drain tube, wherein said stream of urine is collected for disposal. The cotton material f), wherein said cotton material is placed inside said outlet. The valve i), wherein said valve is configured to allow air inside said body, wherein said valve does not allow liquid outside said body.

### DESCRIPTION OF THE DRAWINGS

### References

The description of the figures provided herein contain references to each depicted component. A list of these components described in the context of each figure is provided below for easy reference.
100 Body
102 Lip
104 Adhesive Element
106 Drain Element
108 Fiber Element
110 Outlet
111 Vent
112 Collection Region
113 Valve
114 Fiber Element Support
200 Silicone Layer
500 Attachment Element
600 Underwear

### DESCRIPTION OF THE DRAWINGS

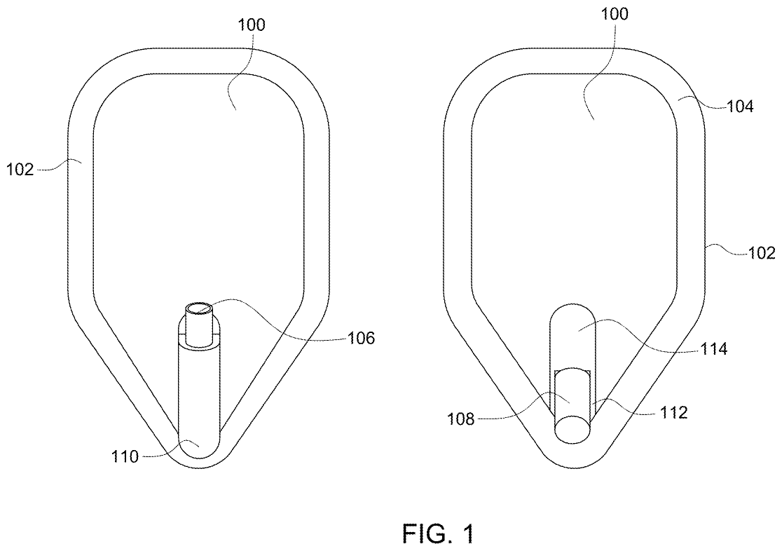

FIG. 1 illustrates the front and back view of the external catheter.

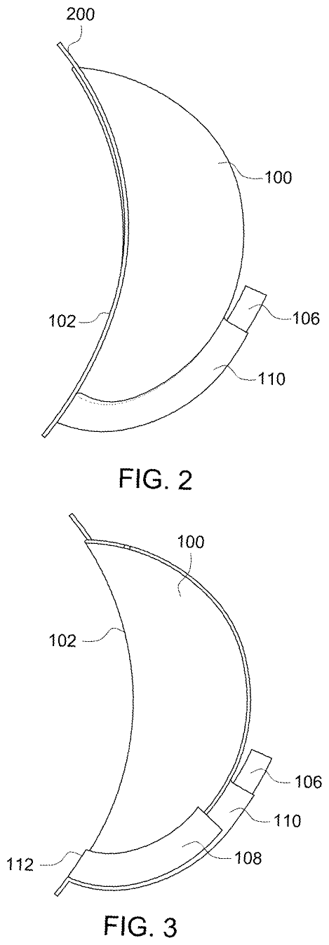

FIG. 2 illustrates the side view of the external catheter.

FIG. 3 illustrates an exploded side view of the external catheter.

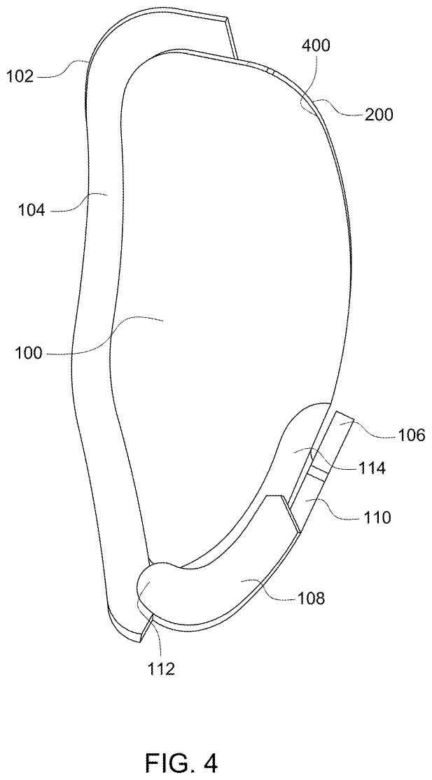

FIG. 4 illustrates a side cross sectional view of the external catheter.

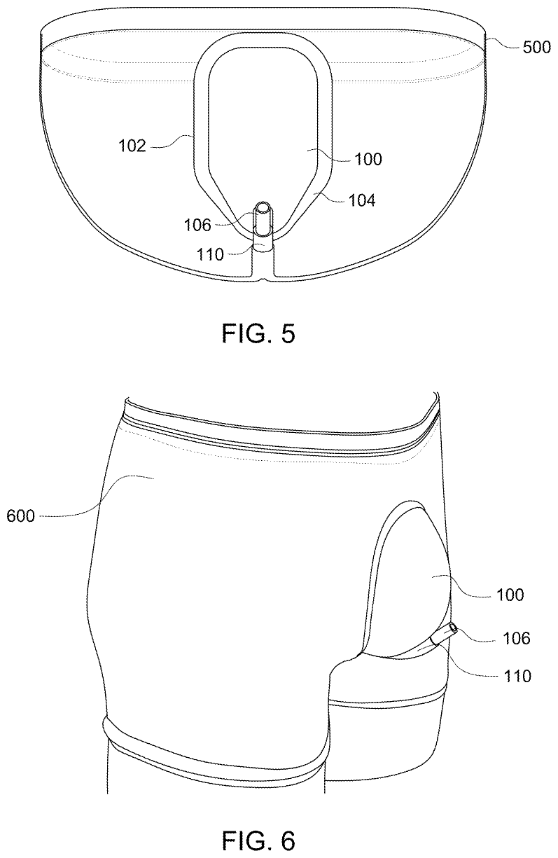

FIG. 5 illustrates the external catheter attached to an attachment element.

FIG. 6 illustrates the external catheter attached to an underwear.

### DETAILED DESCRIPTION

FIG. 1 illustrates the external catheter which can contour to many different types of human bodies having noninvasive capabilities as it is configured in accordance with one or more embodiments of the invention. In this illustrated example, the external catheter comprises a body (100), a lip (102), an adhesive element (104), a drain element (106), a fiber element (108), an outlet (110), a vent (111) a collection region (112), a valve (113) and a fiber element support (114).

The body (100) can be but not limited to a flexible hollow concave non condom structure such that the flexible nature of the structure allows the body (100) to contour or mold to a plurality of human bodies. The human bodies can include women and men of different sizes and shapes. The body (100) of the external catheter covers or encloses a human penis or a human vagina. One end of the external catheter is either left open-ended, to allow drainage into a toilet, or it is attached to a bag to collect the urine. In this embodiment the drain element (106) attached to the body (100), where the drain element can be but is not limited to a drain pipe or a drain tube, or any equivalent structure that it guides the urine to move into an external bag that can be used to collect urine. The drain element (106) is placed inside a harder structure called the outlet (110). The drain element (106) is configured for either prone or standing urine drainage. The outlet (110) holds the drain element (106) in place as well as the fiber element (108) which can be made of cotton, cotton material, or any type of fabric material or any other material that performs the intended purpose. The vent (111) and the valve (113) work in conjunction to allow air from outside the body (100) to come into the body (100) but the valve (113) prevents liquid from exiting the body (100). The valve (113) can be made by a check valve or an extra membrane layer or silicone or any other equivalent structure. The vent (111) is configured to decrease vacuum pressure in the body (100) so that the contents of the body (100) can be voided out with more ease. The function of the fiber element (108) is to guide a stream of urine to the outlet (110). The fiber element (108), outlet and drain element (106) or an equivalent structure are configured to keep urine away from the body (100). In this embodiment the fiber element (108), outlet (110) and drain element (106) or an equivalent structure are each curved and attached to the hallow concave shape of the body (100) of the external catheter. However, before the urine from the user enters the fiber element (108), outlet (110) and drain element (106), it is first collected in collection region (112) at the bottom section of the body (100). The concave shape of the body (100) enables urine to flow downward towards the collection region (112) where urine flows into the fiber element (108), outlet (110) and drain element (106). The fiber element is supported by the fiber support element (114), which allows the fiber element (108) to stay in place and not move around during urination which could cause urine leakage outside the body (100). In addition, the fiber support element (114) covers the interior of the body (100) so in case a human is voiding on a lateral position or any other position, the body (100) will collect liquid and will not allow for leakage of the liquid.

The body (100) is made from but not limited to a lip (102) which forms the border of the body (100). Attached to the lip (102) is an optional layer called an adhesive element (104). The adhesive element (104) could be but is not limited to an adhesive border or an adhesive strip or an adhesive perimeter or any equivalent structure that performs the intended purpose. The adhesive element (104) can be made from but not limited to a Mepilex brand or an equivalent material that performs the same intended purpose. The purpose of the adhesive element (104) is to create a flat surface around the lip (102) of the body (100) such that the body (100) is able to secure to a surface such as a human body while preventing urine leakage during urination.

Traditional catheters are inserted into a penis or a vagina, to drain, of fluids such as urine and therefore can be classified as an invasive since invasive procedures are where deliberate access to the body is gained via an incision, percutaneous puncture, or where medical instruments are used in addition to the puncture needle, or instrumentation via a natural orifice. The external catheter disclosed here does not require any incision, puncture, or any type of invasive methods. Instead, the human penis or vagina sit inside the concave body (100) of the external catheter. When there is urination, the urine will flow downward towards the collection region (112) where urine is collected and subsequently sent down via gravity into the fiber element (108), outlet (110) and drain element (106). The drain element (106) can be connected to a bag or an equivalent structure where urine can be collected.

FIG. 2 illustrates the side view of the external catheter in accordance with one or more embodiments of the invention. In this illustrated example, the external catheter comprises a body (100), a lip (102), a drain element (106), an outlet (110), a vent (111), a valve (113), and a silicone layer (200).

The side view of the body of the external catheter illustrates the concave shape of the body (100) as well as the thickness of the lip (102) which is around the body (100). Also shown in the figure is the outlet (110) which encapsulates the drain element (106). The body (100) of the external catheter is elastic such that it can contour to different humans with different shapes and sizes. Also shown in FIG. 2 is the silicone layer (200) of the body (100). The body (100) can be made from one or more silicone layers because the optional additional silicone layers provide additional center thickness of the body (100), where the extra center thickness keeps urine away from the body (100). In addition, the additional thickness at the center of body (100) keeps the fiber element (108) and urine elevated away from the human user. Silicone is a polymer made up of siloxane and can vary in consistency from liquid to gel to rubber to hard plastic depending on how much layering is present. The vent (111) and the valve (113) work in conjunction to allow air to come into the body (100) but prevent liquid from exiting the body (100). The vent (111) is located on the exterior of the body (100) and the valve (113) is attached to the vent (111). The single silicone layer (200) enables the body (100) to be flexible due to the elastic characteristic of silicone and allows the body (100) to contour to different body types.

FIG. 3 illustrates a transparent side view of the external catheter in accordance with one or more embodiments of the invention. In this illustrated example, the external catheter comprises a body (100), a lip (102), a drain element (106), a fiber element (108), an outlet (110), a vent (111), a collection region (112) and a valve (113).

In this side view of the body (100) of the external catheter, the internal elements of the external catheter can be seen. For example, the fiber element (108) is located inside the outlet (110) and abuts to the collection region (112) on one end and also abuts to the drain element (106) on the other end. In addition, the fiber element (108) which can be but is not limited to cotton, or a section of a fabric material or any equivalent material that serves the intended purpose, is connected to the drain element (106). The outlet (110), fiber element (108) and the drain element (106) each form a curve structure that sits on the surface of the concave body (100) of the external catheter. Also shown in FIG. 3 is the lip (102) of the body (100). The lip (102) attaches to an optional adhesive element (104). The lip (102) and the adhesive element (104) enable a more secure contact between the body (100) of the external catheter and the human user.

FIG. 4 illustrates a cross-section side view of the external catheter in accordance with one or more embodiments of the invention. In this illustrated example, the external catheter comprises a body (100), a lip (102), a drain element (106), a fiber element (108), an outlet (110), a vent (111), a collection region (112), a valve (113), a fiber support element (114), and a silicone layer (200),

FIG. 5 illustrates an external catheter inside an attachment element (500) in accordance with one or more embodiments of the invention. In this illustrated example, the external catheter and an attachment element comprises a body (100), a lip (102), a drain element (106), an outlet (110), a vent (111), a valve (113), and an attachment element (500).

In this embodiment the body (100) of the external catheter is inside an attachment element (500) such that the attachment element (500) holds the body (100) in place and also allows the user to wear the attachment element (500). The attachment element (500) ties around the user's waist and enables the user to put their legs through the bottom section of the attachment element (500), where the attachment element securely fits around the user's waist and legs. The body (100) is positioned in the attachment element (500) such that the concave body (100) of the external catheter fits over the user's penis or vagina. The attachment element (500) is configured for either prone or standing urine drainage. The attachment element (500) allows for the external catheter to stay in place while the user is walking, siting, or going up stairs or any other equivalent action requiring movement. The attachment element (500) can be adjustable such that it can be configured to fit around different people of different shapes and sizes. The adjustment can be accomplished by velcro, or elastic snappable velcro with different notches for different waist sizes or a belt like material with adjustable notches or any equivalent structure that performs the adjustability function.

FIG. 6 illustrates an external catheter attached to an underwear (600) in accordance with one or more embodiments of the invention. In this illustrated example, the external catheter and an underwear (600) comprises a body (100), a drain element (106), an outlet (110), a vent (111), a valve (113), and the attachment element (500).

In this embodiment the body (100) of the external catheter is attached to an underwear (600) such that the underwear (600) and the attachment element (500) work in conjunction to hold the body (100) of the catheter in place and also allows the user to wear the underwear (600). The underwear (600) could be made for either for men or women. Underwear (600) could also be but not limited to shorts, men undergarment, pantyhose, women's undergarments, or any equivalent clothing that performs the intended purpose of an underwear (600). The body (100) is positioned in the underwear (600) such that the concave body (100) of the external catheter fits over the user's penis or vagina. The underwear (600) and the external catheter in this embodiment is configured for either prone or standing urine drainage. The underwear (600) allows for the external catheter to stay in place while the user is walking, siting, or going up stairs.

What is claimed is:

1. An external catheter comprising:
a body, wherein said body is a hollow concave shape;
a lip, wherein said lip is attached around perimeter of said body;
a silicone layer, wherein said silicone layer is a single layer of said body;
a drain element, wherein said drain element is configured for urine drainage; an outlet, wherein said outlet covers said drain element;
a fiber element, wherein said fiber element is attached to said body, wherein said fiber element abuts to drain element;
a collection region, wherein said collection region abuts to said fiber element;
a vent, wherein said vent abuts to said body; and
a valve, wherein said valve is attached to said vent.

2. The external catheter of claim 1, wherein said external catheter is unisex.

3. The silicone layer of claim 1, wherein said silicone layer enables said body to be elastic, wherein said elasticity enables said body to contour to a plurality of human bodies.

4. The collection region of claim 1, wherein said fiber element guides a stream of urine to said drain element, wherein said stream of urine is collected for disposal.

5. The valve of claim 1, wherein said valve is configured to allow air inside said body, wherein said valve does not allow liquid outside said body.

6. An external catheter comprising:
a body, wherein said body is a hollow concave shape;
a lip, wherein said lip is attached around perimeter of said body;
an adhesive element, wherein said adhesive element is attached to said lip, wherein said lip and said adhesive element form a border such that said border enables a sealed attachment to a human body;
a silicone layer, wherein said silicone layer is a single layer of said body;
a drain element, wherein said drain element is configured for urine drainage; an outlet, wherein said outlet covers said drain element;
a fiber element, wherein said fiber element is attached to said body, wherein said fiber element abuts to drain element;
a collection region, wherein said collection region abuts to said fiber element;
a vent, wherein said vent abuts to said body; and
a valve, wherein said valve is attached to said vent.

7. The external catheter of claim 6, wherein said external catheter is unisex.

8. The silicone layer of claim 6, wherein said silicone layer enables said body to be elastic, wherein said elasticity enables said body to contour to a plurality of human bodies.

9. The collection region of claim 6, wherein said fiber element guides a stream of urine to said drain element, wherein said stream of urine is collected for disposal.

10. The adhesive element of claim 6, wherein said adhesive element prevents urine from leaking outside said body during urination.

11. The valve of claim 6, wherein said valve is configured to allow air inside said body, wherein said valve does not allow liquid outside said body.

12. An external catheter comprising:
a body, wherein said body is a hollow concave shape;
a lip, wherein said lip is attached around a perimeter of said body;
a silicone layer, wherein said silicone layer is a single layer of said body; a drain tube, wherein said drain tube is configured for urine drainage;
an outlet, wherein said outlet covers said drain tube;
a cotton material, wherein said cotton material is attached to said body, wherein said cotton material abuts to drain tube;
a collection region, wherein said collection region abuts to said cotton material;
a vent, wherein said vent abuts to said body;
a valve, wherein said valve is attached to said vent; and
an attachment element, wherein said attachment element is configured to hold said body, wherein said attachment element is adjustable.

13. The external catheter of claim 12, wherein said external catheter is unisex.

14. The silicone layer of claim 12, wherein said silicone layer enables said body to be elastic, wherein said elasticity enables said body to contour to a plurality of human bodies.

15. The collection region of claim 12, wherein said cotton material guides a stream of urine to said drain tube, wherein said stream of urine is collected for disposal.

16. The cotton material of claim 12, wherein said cotton material is placed inside said outlet.

17. The valve of claim 12, wherein said valve is configured to allow air inside said body, wherein said valve does not allow liquid outside said body.

18. An external catheter comprising:
a body, wherein said body is a hollow concave shape;
a lip, wherein said lip is attached around a perimeter of said body;
a silicone layer, wherein said silicone layer is a single layer of said body;
a drain tube, wherein said drain tube is configured for urine drainage; an outlet, wherein said outlet covers said drain tube;
a cotton material, wherein said cotton material is attached to said body, wherein said cotton material abuts to drain tube;
a collection region, wherein said collection region abuts to said cotton material;
a vent, wherein said vent abuts to said collection region;
a valve, wherein said valve is attached to said drain element;
an attachment element, wherein said attachment element is configured to hold said body, wherein said attachment element is adjustable; and
an underwear, wherein said underwear is configured with said attachment element to hold said body, wherein said underwear is unisex.

19. The silicone layer of claim 18, wherein said silicone layer enables said body to be elastic, wherein said elasticity enables said body to contour to a plurality of human bodies.

20. The collection region of claim 18, wherein said cotton material guides a stream of urine to said drain tube, wherein said stream of urine is collected for disposal.

21. The cotton material of claim 18, wherein said cotton material is placed inside said outlet.

22. The valve of claim 18, wherein said valve is configured to allow air inside said body, wherein said valve does not allow liquid outside said body.
